(19) United States
(12) Patent Application Publication
(10) Pub. No.: US 2024/0228736 A1
(43) Pub. Date: Jul. 11, 2024

(54) A Process for Preparing a Monoester of Terephthalic Acid and Its Derivatives

(71) Applicant: Agency for Science, Technology and Research, Singapore (SG)

(72) Inventor: He-Kuan Luo, Singapore (SG)

(21) Appl. No.: 18/558,643

(22) PCT Filed: Jan. 6, 2022
(86) PCT No.: PCT/SG2022/050006

§ 371 Date: Nov. 2, 2023

(30) Foreign Application Priority Data
Jan. 7, 2021 (SG) 10202100177W

(51) Int. Cl. C08J 11/24; C07C 51/09; C07C 67/03; C07C 67/30

(52) U.S. Cl. CPC C08J 11/24; C07C 51/09; C07C 67/03; C07C 67/30; C08J 2367/02

(57) ABSTRACT

The present invention relates to processes for depolymerizing a polyester of a dicarboxylic acid and for hydrolyzing a diester of terephthalic acid in the presence of a first base and a solvent mixture into a mono-salt of a monoester of the dicarboxylic acid and further comprises the further treatment of said mono-salt product into other useful derivatives, wherein the solvent mixture comprises at least one aprotic solvent and at least one protic solvent; and wherein the aprotic solvent and protic solvent are provided in a volume ratio of from 1:10 to 100:1. In a preferred embodiment, the aprotic solvent is dichloromethane (DCM), acetonitrile (ACN), tetrahydrofuran (THF) or diethyl ether; and the protic solvent is methanol, ethanol, or allyl alcohol; and the base is potassium hydroxide (KOH).

### CROSS-REFERENCE TO RELATED APPLICATIONS

This application claims the benefit of priority of Singapore patent application No. 102021100177W, filed on 7 Jan. 2021, its contents being hereby incorporated by reference in its entirety.

### TECHNICAL FIELD

The present invention relates to the depolymerization of polyester waste to produce a monoester of dicarboxylic acid and other useful products.

### BACKGROUND ART

Polyester is a class of polymers which contain ester functional groups in every repeat unit at the polymer backbone. Among the family of polyesters, terephthalate polyesters, i.e. poly(alkylene terephthalate)s, particularly polyethylene terephthalate (PET) and polybutylene terephthalate (PBT), are the most common thermoplastic resins, which are widely used in the manufacturing of various bottles (for drinking water, soft drinks, body soaps and shampoos), boxes (for foods and fruits packing), barrier films, fibres and textiles. Therefore, there is a large volume of PET and PBT waste generated every year. Such polyester waste, especially waste PET scraps, has to be recycled for environmental protection.

PET is a high molecular weight (MW) polymer and conversion of PET back to its monomers is usually a slow process, sometimes requiring a high temperature, and which typically results in a mixture of multiple end-products. In PET recycling or upcycling processes, one of the key technical problems is selective depolymerization of waste PET to produce desired monomers or value-added chemicals. However, the state-of-the-art technologies are currently limited to hydrolysis to terephthalic acid (TPA), methanolysis to dimethyl terephthalate (DMT), and glycolysis to bis-(2-hydroxyethyl) terephthalate (BHET). These three products (TPA, DMT, BHET) have different functional groups on the phenyl ring. But for each one of them, it has the same alkoxy group (RO) at 1,4-positions of phenyl.

In other embodiments, there has been disclosed a depolymerization method for converting waste PET to dipotassium salt of TPA, which comprises a typical hydrolysis of PET. The aqueous solution of dipotassium salt of TPA was acidified with aqueous sulfuric acid to produce TPA monomers. The depolymerization was carried out with potassium hydroxide or sodium hydroxide using methanol as major solvent (95-97 vol %), and a non-polar solvent dichloromethane as co-solvent (3-5 vol %) at room temperature. Typical volume ratios of the non-polar solvent to alcohol may be from about 1:10 to about 1:50.

Recently, a low-energy catalytic methanolysis of PET was reported to produce DMT promoted by potassium carbonate at a mild temperature range of 20-35° C. in a solvent mixture of methanol and dichloromethane. Potassium carbonate is an inexpensive and non-toxic base. However, the depolymerization rate is slow, and may take up to or more than 24 hours.

In another known method, PET is depolymerized by glycolysis promoted using an amidine organocatalyst, such as 1,8-diazabicycloundec-7-ene (DBU), at high temperatures up to 190° C. The depolymerization reaction resulted in a mixture containing bis(2-hydroxyethyl)terephthalate (BHET). However, this depolymerization method is more energy intensive compared to the hydrolysis or methanolysis at mild conditions.

So far, there is no report to convert waste PET to monoester of terephthalic acid, which has different groups at 1,4-positions of phenyl.

Monoesters of terephthalic acid are useful fine chemicals in a variety of applications including polymer synthesis and pharmaceutical industry. These applications usually require the chemicals to be in a highly pure form (e.g., >99 wt. %). Monoesters of terephthalic acid are known as co-products in the production of oxidized products from para-xylene or as a product from hydrolysis of terephthalate derivatives. However, the separation of the monoesters from such reaction product mixtures is known to be difficult. Therefore, despite its wide applications, the cost of producing monoesters of terephthalic acid remains relatively high compared to other terephthalic derivatives.

Generally, diesters of terephthalic acid can serve as a raw material for the production of monoesters of terephthalic acid. Conventionally, to obtain monoesters of terephthalic acid, diesters of terephthalic acid are hydrolyzed using selective enzymes or metal salt catalysts. However, these known methods suffer from several disadvantages, for instance, the hydrolysis products inevitably contain a mixture of monoesters of terephthalic acid and terephthalic acid. Moreover, the reaction is typically slow (e.g., may take up to 24 hours) and requires elevated temperatures. Moreover, these methods usually result in low yield of the monoesters (<70%).

In one known method, potassium salt of monomethyl terephthalate was prepared from DMT in a solvent mixture of benzene and ethanol at 55° C., or in methanol at refluxing temperature. The aqueous solution of potassium salt of monomethyl terephthalate was acidified with sulfuric acid to produce monomethyl terephthalate. This monohydrolysis method was run at 55° C. or refluxing temperature in methanol, which consumes more energy compared to reactions run at room temperature. Another disadvantage with this method is the use of benzene, which is an aromatic carcinogenic solvent.

In other methods, terephthalic acid has been used as a raw material for producing monoesters of terephthalic acid. For such methods, alumina catalysts are typically used to protect one of the two carboxylic groups of terephthalic acid from reacting with methylating reagent. However, these methods nonetheless suffer from limited yield and selectivity of the desired monoester products. While a high selectivity may be reached by adjusting concentration of reactants, such high selectivity typically results in a trade-off of having very low conversion of product (e.g., 0.1 wt. %).

Hence, there is a need to provide a novel solution to process polyester waste, in particular PET waste, to produce useful products such as monoesters of terephthalic acid with improved yield and selectivity.

### SUMMARY OF INVENTION

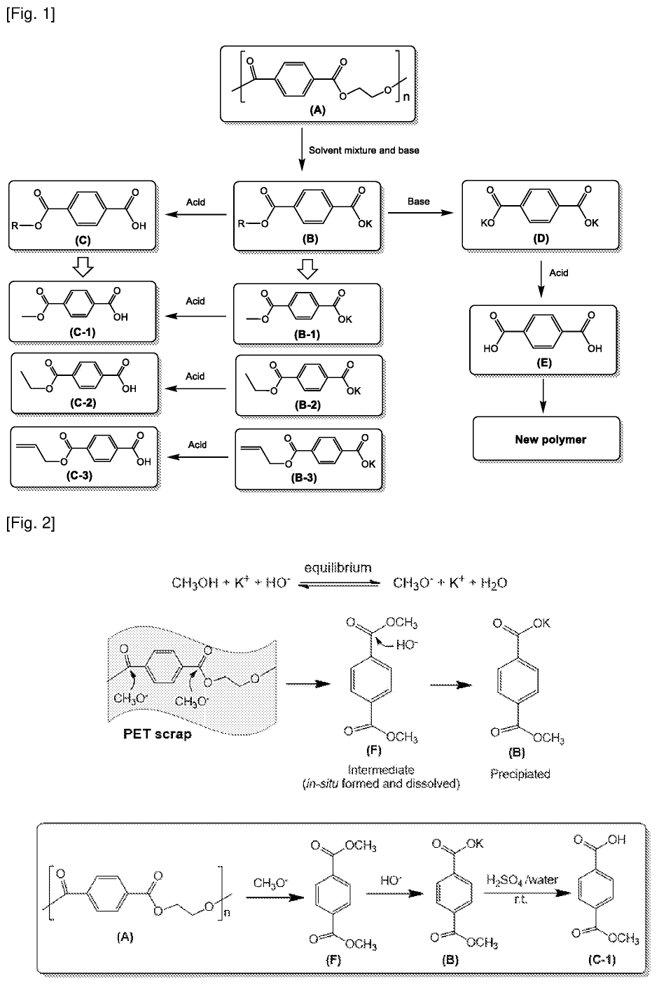

There is provided a process of depolymerizing a polyester of a dicarboxylic acid, the process comprising depolymerizing the polyester in the presence of a first base and a solvent mixture to yield a product comprising a mono-salt of a monoester of dicarboxylic acid (e.g., FIG. 1); wherein said solvent mixture comprises at least one aprotic solvent and at least one protic solvent; and wherein the volume ratio of aprotic solvent and protic solvent is from 1:10 to 100:1.

Advantageously, the provision of at least one protic solvent and at least one aprotic solvent is found to surprisingly improve the selectivity, yield, and efficiency for the process. In particular, the processes disclosed herein may provide yields of up to 84% and selectivities of up to 99.3%. Additionally, the processes disclosed herein may be undertaken under ambient conditions with a time interval of 0.5-5 hours.

It has been unexpectedly found that the use of the described solvent mixture allows a one-pot production of the mono-salt of monoester of dicarboxylic acid from polyesters with superior yield and selectivity. In particular, the solvent mixture may be adjusted to provide optimal concentrations of protic solvents and bases in the reaction mixture. The optimal concentrations of protic solvents and bases enable the in-situ formation of the diester of dicarboxylic acid and its immediate dissolution to be converted to mono-salt of dicarboxylic acid (e.g., FIG. 2). Furthermore, the solvent mixture may effectively precipitate the mono-salt of monoester of dicarboxylic acid once it is formed, which drives the depolymerization preferentially toward the mono-salt product, thereby improving the yield and selectivity. Further advantageously, the disclosed process which yields the mono-salt product may be readily used to upcycle polyester waste into valuable fine chemicals or monomers for producing new polymers.

In another aspect of the disclosure, there is also provided a process for preparing a mono-salt of monoester of terephthalic acid, the process comprising: hydrolysing a diester of terephthalic acid in the presence of a first base and a solvent mixture to yield a mono-salt of the monoester of terephthalic acid with selectivity of up to 99.9%; wherein said solvent mixture comprises at least one protic solvent and at least one aprotic solvent; and wherein the aprotic solvent and protic solvent are provided in a volume ratio of from 1:10 to 100:1.

### Definitions

The following words and terms used herein shall have the meaning indicated: The term “polyester” is to be interpreted broadly to include any polymers that comprise ester functional group.

The term ‘terephthalate polyester’ is to be interpreted broadly to include any polymers that comprise terephthalate blocks.

The term “terephthalic polymer waste” is to be interpreted broadly to include any wastes of textiles, packaging, tapes, flexible electronics, cables that contain terephthalate polymers. Examples may include but not limited to waste cleaning cloths, waste of solar cell substrate, waste PET (polyethylene terephthalate) bottles of water, waste PET bottles of soft drinks, coloured waste PET bottles of shampoos, colored waste PET bottles of body soaps, waste PET boxes of fruits, industry PET waste, PET films, multi-layered films of PET/adhesive/PE, or a combination thereof.

The term “alcoholysis” refers to the process of a chemical reaction that occurs between an organic molecule and an alcohol. E.g. transesterification is a kind of alcoholysis, in which the alcohol from an ester is displaced by another alcohol.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means+/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### LIST OF ABBREVIATIONS

PET: polyethylene terephthalate
K-MMT: potassium salt of monomethyl terephthalate
MMT: monomethyl terephthalate
K-MET: potassium salt of monoethyl terephthalate
MET: monoethyl terephthalate
K-MALT: potassium salt of monoallyl terephthalate
MALT: monoallyl terephthalate
DMT: dimethyl terephthalate
K₂-TPA: dipotassium salt of terephthalic acid
TPA: terephthalic acid
DCM: dichloromethane
THF: tetrahydrofuran
ACN: acetonitrile

### Detailed Disclosure of Embodiments

Exemplary, non-limiting embodiments of a process of depolymerizing a polyester of a dicarboxylic acid, will now be disclosed.

In one embodiment, there is provided a process of depolymerizing a polyester of a dicarboxylic acid. The process may comprise depolymerizing the polyester in the presence of a first base and a solvent mixture to yield a product comprising a mono-salt of monoester of dicarboxylic acid (e.g., FIG. 1); wherein said solvent mixture comprises at least one aprotic solvent and at least one protic solvent; and wherein the aprotic solvent and protic solvent are provided in a volume ratio from 1:10 to 100:1.

The polyester may be produced from monomers comprising dicarboxylic acid and diol or dicarboxylic acid-diol oligomer.

The polyester may be selected from the group consisting of aliphatic polyester, aromatic polyester, block copolymer of forgoing polyester, branched foregoing polyesters, substituted foregoing polyesters and mixtures thereof.

The aliphatic polyester may be selected from the group consisting of polyethylene adipate (PEA), polybutylene succinate (PBS), substituted foregoing polyesters, block copolymer of forgoing polyesters, branched forgoing polyesters and mixtures thereof.

The aromatic polyester may be selected from the group consisting of polyethylene terephthalate (PET), polybutylene terephthalate (PBT), polytrimethylene terephthalate (PTT), polyethylene naphthalate (PEN), substituted foregoing polyesters, block copolymer of forgoing polyesters, branched forgoing polyesters and mixtures thereof.

The polyester may be in a waste form.

In the depolymerizing process, diester of dicarboxylic acid may be an intermediate before the precipitation of said mono-salt via alcoholysis, wherein the organic group of the protic solvent will be incorporated into the two terminals of dicarboxylic block to form a diester of the dicarboxylic acid.

The polyester may be provided in a concentration of from 0.1 M to 2 M, or 0.1 M to 1.5 M, or 0.1 M to 1 M. The polyester concentration is calculated by the repeating monomer unit (i.e., dicarboxylic acid or diol) in mole divided by the volume of the solution.

The first base may comprise at least one inorganic base, at least one organic base or mixtures thereof. The inorganic base may be selected from the group consisting of an alkali metal hydroxide, an alkaline earth metal hydroxide, an ammonium hydroxide, and mixtures thereof. More preferably, said inorganic base may be potassium hydroxide, sodium hydroxide, and mixtures thereof. In embodiments, the base is potassium hydroxide, or sodium hydroxide.

The first base may be selected from the group consisting of: a metal salt of methoxide, a metal salt of ethoxide, a metal salt of n-propoxide, a metal salt of iso-propoxide, a metal salt of n-butoxide, a metal salt of tert-butoxide, and mixtures thereof, wherein the metal may be selected from an alkali metal or an alkaline earth metal.

The first base may also be selected from the group consisting of: potassium methoxide, potassium ethoxide, potassium n-propoxide, potassium iso-propoxide, potassium n-butoxide, potassium tert-butoxide, sodium methoxide, sodium ethoxide, sodium n-propoxide, sodium iso-propoxide, sodium n-butoxide, sodium tert-butoxide, and mixtures thereof.

The protic solvent may be selected from the group consisting of methanol, ethanol, 2-fluoroethanol, 2-chloroethanol, 2,2,2-trichloroethanol, n-propanol, isopropanol, n-butanol, tert-butanol, allyl alcohol, propargyl alcohol, 2-aminoethanol, 2-dimethylaminoethanol, ethylene glycol, propylene glycol, 1,4-butanediol, and mixtures thereof. In embodiments, the protic solvent is selected from methanol, ethanol or allyl alcohol.

The aprotic solvent disclosed may be selected based on the following requirements: 1) the aprotic solvent can swell the polyester better than the protic solvent; 2) the diester of dicarboxylic acid, which is in-situ generated from the corresponding polyester via alcoholysis, has a better solubility in the aprotic solvent than in the protic solvent; 3) the aprotic solvent is miscible with the protic solvent at any volume ratio; 4) the mono-salt of monoester of dicarboxylic acid has a poorer solubility in the aprotic solvent than in the protic solvent.

The aprotic solvents may be selected from a group consisting of a polar aprotic solvent, a non-polar aprotic solvent and combinations thereof.

The polar aprotic solvent may be selected from the group consisting of acetonitrile, tetrahydrofuran, acetone, dimethyl formamide, dimethyl sulfoxide and combinations thereof.

The non-polar aprotic solvent may be selected from the group consisting of toluene, dichloromethane, chlorobenzene, xylene, diethyl ether and combinations thereof.

The preferred aprotic solvents used in the embodiments are acetonitrile, tetrahydrofuran, dichloromethane, or diethyl ether.

Compared to the use of pure protic solvents, the solvent mixture comprising at least one aprotic solvent and at least one protic solvent may advantageously reduce the likelihood of the formation of hydrogen bonds between protic solvents and ester groups of polyester or the intermediate diester of dicarboxylic acid, thus reducing the spatial hinderance for the depolymerizing process.

Advantageously, the solvent mixture may reduce the concentration of protic solvent molecules and dissolve the base at a slower speed compared to the dissolution rate of base in protic solvent only. On the one hand, compared to pure protic solvents, the decreasing concentration of protic solvent reduces side reactions with the base and provides more chances for the base to approach the carbonyl group of the in-situ formed diester for the formation of the mono-salt. On the other hand, the slower dissolution rate of the base also leads to a bigger molar ratio between the intermediate diester of dicarboxylic acid and base, which afforded an unexpectedly fast conversion and high selectivity towards the monohydrolysis of the in-situ generated diester of dicarboxylic acid to form the mono-salt of the monoester of dicarboxylic acid.

Also advantageously, although polyesters, such as PET, are not soluble in the organic solvent mixture, the ester groups of polyester have better interaction with the organic solvent mixture compared to using protic solvent only. That is, organic solvent mixture can swell polyesters better. Therefore, polyesters contact well with the base in the solvent mixture to facilitate the depolymerizing step with a higher concentration of reactant, which is beneficial to a faster depolymerization reaction via alcoholysis to produce the corresponding diester of dicarboxylic acid.

In addition, the in-situ produced diester of dicarboxylic acid has better solubility in the organic solvent mixture compared to using protic solvent only, which provides higher concentration of reactants for faster monohydrolysis to produce the mono-salt of monoester of dicarboxylic acid.

Furthermore, the mono-salt as formed tends to be precipitated immediately from the reaction solution, which preferentially leads to the desired mono-salt product by minimizing further hydrolysis of mono-salt product to a dicarboxylic salt.

Advantageously, the constant conversion of the intermediate diester of dicarboxylic acid to the mono-salt product in the reaction mixture promotes the depolymerization of polyesters.

This depolymerization process is a novel concurrent procedure comprising alcoholysis of polyesters and monohydrolysis of the in-situ generated diester of dicarboxylic acid in one pot (e.g., FIG. 2).

It is believed that by optimizing the concentrations of the protic solvent and the base in the reaction, the diester of dicarboxylic acid as formed in-situ in the depolymerizing of polyester may be immediately converted to a mono-salt product, thus creating a novel concurrent alcoholysis and monohydrolysis procedure to convert polyester waste selectively and efficiently to mono-salt of monoester of dicarboxylic acid in a one-pot reaction (e.g., FIG. 2).

Where the polyester is colored, the disclosed process may further comprise a step of decolorizing the polyester in the presence of organic solvents prior to its depolymerization. The organic solvents may be selected from the group consisting of dichloromethane, tetrahydrofuran, acetonitrile, dimethyl sulfoxide and mixtures thereof. The decolorization process may be completed in a time duration ranging from about 1 hour to about 24 hours. The decolorization process may be performed in a temperature ranging from about 25° C. to about 60° C.

Alternatively, the removal of dyes (i.e. decolorization) in the polyester waste may be carried out after the depolymerization of colored polyesters. In embodiments, the colored polyester is first depolymerized. Then the produced mono-salt will be dissolved into water. Since the organic dyes are not soluble in water, thus they may be removed by filtration, or by centrifugation, or by column chromatography.

The depolymerizing process may be performed at a temperature of about 10° C. to about 90° C., or about 10° C. to about 80° C., or about 10° C. to about 75° C., or about 10° C. to about 65° C., or about 15° C. to about 60° C., or about 20° C. to about 60° C. In embodiments, the depolymerizing process is performed at about 22° C., about 40° C., or about 55° C.

Advantageously, the solvent mixture enables the depolymerizing process to be completed at room temperature or slightly higher temperature above room temperature, whereas, known methods may be undertaken at a temperature of at least 180° C. The low temperature reduces the energy consumption when manufacturing the monoester product.

In the depolymerizing process, the mole ratio of the first base to the repeating monomer unit of polyester may be provided in the range from about 0.5 to about 2.5, or about 0.5 to about 2, or about 0.5 to about 1.5, or about 1 to 1.5. In embodiments, the mole ratio of base to the repeating monomer unit of polyester is about 1, or about 1.2 or about 1.5. It has been found that the disclosed mole ratios of the first base to the repeating monomer unit of polyester, in particular, from 0.5-2 or from 1-1.5, may be useful in obtaining an optimal balance between yield (based on total conversion of the polyester) and the selectivity towards the mono-salt product.

Advantageously, a slight excess of base relative to the repeating monomer unit of polyester in mole basis may accelerate the reaction while still retaining a high selectivity of at least 98%.

The volume ratio of the aprotic solvent and the protic solvent may be adjusted to realize an optimal selectivity and yield towards the mono-salt of dicarboxylic acid with minimal impurities, e.g., dicarboxylic salt.

The volume ratio of the aprotic solvent to the protic solvent in the solvent mixture may be in the range from about 100:1 to about 1:10, about 90:1 to about 1:10, or about 80:1 to about 1:10, or about 70:1 to about 1:10 or about 60:1 to about 1:10, or about 50:1 to about 1:10, or about 40:1 to about 1:10, or about 30:1 to about 1:10, or about 20:1 to about 1:10, about 15:1 to about 1:10, about 10:1 to about 1:10, or about 8:1 to about 1:10, or about 6:1 to about 1:10, or about 4:1 to about 1:10, or about 2:1 to about 1:10, or about 1:1 to about 1:10, or about 1:1 to about 1:8, about 1:1 to about 1:6, or about 1:1 to about 1:4, or about 1:1 to about 1:2.

The depolymerizing process may be performed for a time duration ranging from about 10 min to about 10 hours, or about 10 min to about 8 hours, or about 10 min to about 6 hours, or about 10 min to about 5 hours, or about 20 min to about 5 hours, or about 30 min to about 5 hours. In embodiments, the depolymerizing process is completed within about 0.5 hour, about 1 hour, about 2 hours or about 4 hours.

After the depolymerizing process, the mono-salt solid as precipitated may be filtrated and washed by protic or aprotic solvents for at least 2 times. The mono-salt solid may then be dried in an oven at a temperature of 60° C. The dried mono-salt solid may be dissolved in water to form a solution for further use. The concentration of said mono-salt solution may be between 2-10 wt. %, preferably between 3-5 wt. %.

The depolymerizing process may further comprise acidifying the mono-salt precipitates to produce a monoester of said dicarboxylic acid.

The acidifying step may comprise acidifying the mono-salt solution to form the monoester of dicarboxylic acid by adding an acid into the solution until pH reaches 0.1 to 3 and preferably 1 to 2. The acid may be selected from hydrochloric acid (HCl), sulfuric acid (H₂SO₄), phosphoric acid (H₃PO₄), acetic acid, formic acid, hydrobromic acid, citric acid and mixtures thereof. In embodiments, the acid is H₂SO₄.

The acidifying step as disclosed may be performed in a temperature ranging from about 18° C. to about 40° C. In embodiments, the acidifying step is performed at ambient environment with a temperature of about 22° C.

The monoester product may be precipitated from the acidified solution once formed and thus may be separated from the solution via general filtration. The monoester solid may be dried in an oven under 60° C. The yield of the monoester product may be between 70% and 99% with a purity between 94% and 99%.

The depolymerizing process may further comprise hydrolysing the mono-salt precipitates in the presence of a second base to produce a dicarboxylic salt.

The hydrolysing of the mono-salt solution may be performed at a temperature ranging from about 20° C. to about 40° C. The hydrolysing of the mono-salt solution may be undertaken for a time period of 10 minutes to 1 hour.

In the hydrolysing of the mono-salt, the second base may be selected from the group consisting of an alkali metal hydroxide, an alkaline earth metal hydroxide, an ammonium hydroxide, and mixtures thereof. More preferably, the second base may be potassium hydroxide, sodium hydroxide, and mixtures thereof. In embodiments, the base is potassium hydroxide, or sodium hydroxide.

The mole ratio of the second base to the mono-salt may be provided in a range of 0.5 to 2.5, or about 0.5 to about 2, or about 0.5 to about 1.5, or about 1 to 1.5. In embodiments, the mole ratio of the second base to the mono-salt is about 1.2.

The dicarboxylic salt obtained from the hydrolysing process may undergo an acidifying process to produce a dicarboxylic acid.

The acidifying process may comprise acidifying the dicarboxylic salt solution with an acid to form a dicarboxylic acid, which is thus precipitated once formed. The acid may be selected from hydrochloric acid (HCl), sulfuric acid (H₂SO₄), phosphoric acid (H₃PO₄), acetic acid, formic acid, hydrobromic acid, citric acid and mixtures thereof. The acid disclosed may be added to the dicarboxylic salt solution after the hydrolysing process until pH reaches 0.1 to 3 and preferably 1 to 2. The acidifying step disclosed may be performed at a temperature ranging from about 18° C. to about 40° C.

After the acidifying step, the precipitated dicarboxylic acid may be collected by general filtration and further dried in an oven at 60° C.

In another embodiment, there is also provided a process of preparing a mono-salt of monoester of terephthalic acid. The process may comprise hydrolysing a diester of terephthalic acid in the presence of a first base and a solvent mixture to yield a mono-salt of monoester of terephthalic acid; wherein said solvent mixture comprises at least one aprotic solvent and at least one protic solvent; and wherein the aprotic solvent and protic solvent are provided in a volume ratio of from 1:10 to 100:1.

The first base may comprise at least one inorganic base, at least one organic base or mixtures thereof.

The first base may be selected from the group of inorganic bases consisting of an alkali metal hydroxide, an alkaline earth metal hydroxide, an ammonium hydroxide, and mixtures thereof. More preferably, the first base may be potassium hydroxide, sodium hydroxide, and mixtures thereof. In embodiments, the base is potassium hydroxide, or sodium hydroxide.

The first base may also be selected from the group of organic bases consisting of metal salt of methoxide, metal salt of ethoxide, metal salt of n-propoxide, metal salt of iso-propoxide, metal salt of n-butoxide, metal salt of tert-butoxide, and mixtures thereof, wherein the metal is selected from an alkali metal or an alkaline earth metal.

More preferably, the first base may be selected from the groups of organic bases consisting of potassium methoxide, potassium ethoxide, potassium n-propoxide, potassium iso-propoxide, potassium n-butoxide, potassium tert-butoxide, sodium methoxide, sodium ethoxide, sodium n-propoxide, sodium iso-propoxide, sodium n-butoxide, sodium tert-butoxide, and mixtures thereof.

The protic solvent may be selected from the group consisting of methanol, ethanol, 2-fluoroethanol, 2-chloroethanol, 2,2,2-trichloroethanol, n-propanol, isopropanol, n-butanol, tert-butanol, allyl alcohol, propargyl alcohol, 2-aminoethanol, 2-dimethylaminoethanol, ethylene glycol, propylene glycol, 1,4-butanediol, and mixtures thereof. In embodiments, the protic solvent is methanol, ethanol or allyl alcohol.

The aprotic solvent may be selected to meet the following requirements: 1) diester of terephthalic acid has a better solubility in the aprotic solvent than in the protic solvent; 2) the aprotic solvent is miscible with the protic solvent at any volume ratio; 3) the mono-salt of monoester of terephthalic acid has a poorer solubility in the aprotic solvent than in the protic solvent.

The aprotic solvents may be selected from a group consisting of a polar aprotic solvent, a non-polar aprotic solvent and combinations thereof.

The polar aprotic solvent may be selected from the group consisting of acetonitrile, tetrahydrofuran, acetone, dimethyl formamide, dimethyl sulfoxide and combinations thereof.

The non-polar aprotic solvent may be selected from the group consisting of toluene, dichloromethane, chlorobenzene, xylene, diethyl ether and combinations thereof.

In embodiments, the aprotic solvent may be selected from acetonitrile, tetrahydrofuran, dichloromethane and diethyl ether.

The diester may be provided in a concentration of from 0.1 M to 2 M, or 0.1 M to 1.5 M, or 0.1 M to 1 M.

The hydrolysing of the diester may be performed at a temperature of about 10° C. to about 90° C., or about 10° C. to about 80° C., or about 10° C. to about 75° C., or about 10° C. to about 65° C., or about 15° C. to about 60° C., or about 20° C. to about 60° C. In embodiments, the hydrolysing step is performed at about 22° C., about 40° C., or about 55° C.

The mole ratio of the first base to the diester of terephthalic acid may be in the range from about 0.5 to about 2.5, or about 0.5 to about 2, or about 0.5 to about 1.5, or about 1 to 1.5. In embodiments, the mole ratio of base to the diester of terephthalic acid is about 1, or about 1.2 or about 1.5.

The volume ratio of the aprotic solvent to the protic solvent in the organic solvent mixture may be in the range from about 100:1 to about 1:10, about 90:1 to about 1:10, or about 80:1 to about 1:10, or about 70:1 to about 1:10 or about 60:1 to about 1:10, or about 50:1 to about 1:10, or about 40:1 to about 1:10, or about 30:1 to about 1:10, or about 20:1 to about 1:10, about 15:1 to about 1:10, about 10:1 to about 1:10, or about 8:1 to about 1:10, or about 6:1 to about 1:10, or about 4:1 to about 1:10, or about 2:1 to about 1:10, or about 1:1 to about 1:10, or about 1:1 to about 1:8, about 1:1 to about 1:6, or about 1:1 to about 1:4, or about 1:1 to about 1:2.

The hydrolysing of the diester may be completed in a time duration ranging from about 10 minutes to about 10 hours, or about 10 minutes to about 8 hours, or about 10 minutes to about 6 hours, or about 10 minutes to about 4 hours, or about 20 minutes to about 4 hours, or about 30 minutes to about 4 hours. In embodiments, the hydrolysing step is completed within about 0.5 hour, about 1 hour, about 2 hours or about 4 hours.

After the hydrolysing of the diester, the precipitated mono-salt solid may be filtrated and washed by protic or aprotic solvents for at least 2 times. The solid may then be dried in an oven at a temperature of 60° C. The dried solid may be dissolved in water to obtain an aqueous mono-salt with a concentration between 2-10 wt. %, preferably between 3-5 wt. %.

### BRIEF DESCRIPTION OF DRAWINGS

The accompanying drawings illustrate a disclosed embodiment and serve to explain the principles of the disclosed embodiment. It is to be understood, however, that the drawings are designed for purposes of illustration only, and not as a definition of the limits of the invention.

### FIG. 1

FIG. 1 depicts the conversion of waste PET to monoesters of terephthalic acid and TPA monomers.

### FIG. 2

FIG. 2 depicts the mechanism of concurrent methanolysis and monohydrolysis of PET.

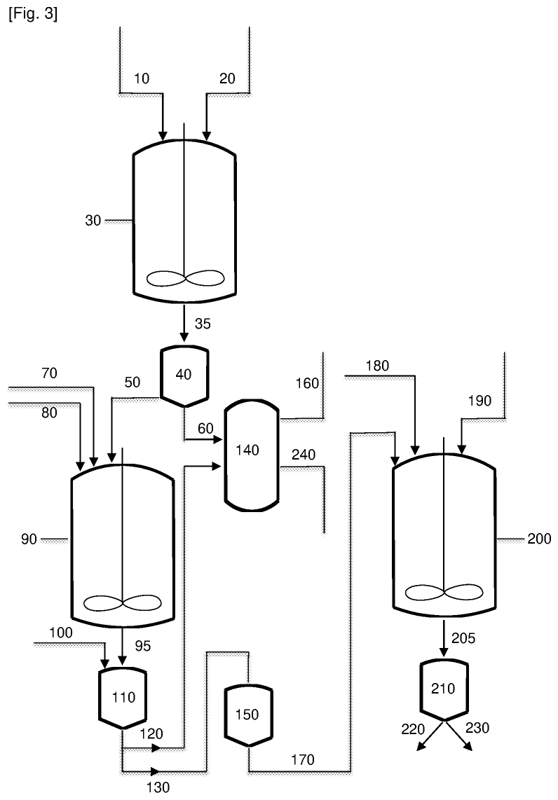

### FIG. 3

FIG. 3 is a chemical flow chart depicting a large-scale production of monoester of dicarboxylic acid and its derivative dicarboxylic acid from polyester waste.

### DETAILED DESCRIPTION OF DRAWINGS

### Detailed Description of [FIG. 1]

Referring to FIG. 1, there is shown a process of depolymerizing PET waste in the presence of at least a first base and a solvent mixture comprising at least one aprotic solvent and at least one protic solvent. When methanol is used as the protic solvent together with an aprotic solvent, PET is converted to K-MMT(B-1), which is then acidified to produce MMT (C-1). K-MMT can be further hydrolyzed to K₂-TPA (D) in the presence of a second base, which is then acidified to produce TPA (E). When ethanol is used as the protic solvent together with an aprotic solvent, PET is converted to K-MET(B-2), which is then acidified to produce MET (C-2). When allyl alcohol is used as the protic solvent together with an aprotic solvent, PET is converted to K-MALT(B-3), which is then acidified to produce MALT (C-3). It is to be understood that the first base, the second base and the protic solvent disclosed in the description may be applied in the process as depicted in FIG. 1.

### Detailed Description of [FIG. 2]

Referring to FIG. 2, the mechanism of concurrent methanolysis and monohydrolysis in a one-pot procedure to directly convert waste PET to K-MMT (B) is depicted. The one-pot procedure refers to a concurrent alcoholysis-monohydrolysis process, wherein the polyester undergoes an alcoholysis process by the protic solvent (generally an alcohol) together with aprotic solvents, followed by an immediate monohydrolysis by the base. Here in FIG. 2, methanol is used as the protic solvent together with an aprotic solvent. When KOH is dissolved into this solvent mixture, an equilibrium is built up to form a solution containing both methoxide anions (CH₃O⁻) and hydroxyl anions (HO⁻). Since CH₃O⁻ anion is a stronger base compared to HO⁻ anion, CH₃O⁻ anion will first attack the carbon of C═O on the surface of solid PET to in-situ generate DMT (F). The in-situ generated DMT is very soluble in the solvent mixture, thus one of the two ester groups of DMT will be hydrolyzed by HO⁻ anion to form K-MMT, which is a monohydrolysis reaction. The produced K-MMT will precipitate out immediately from the solution because it has poor solubility in the solvent mixture. The K-MMT can be acidified with H₂SO₄ to produce MMT (C-1) readily in water at room temperature.

### Detailed Description of [FIG. 3]

Referring to FIG. 3, there is shown a decolorizing reactor unit 30 which is configured to receive an organic solvent 10 and a polyester waste 20 for decolorizing existing colours in the polyester waste. The decolorizing is undertaken by stirring with a mechanical stirrer at room temperature (22° C.) to obtain a mixture of coloured solvent and decolorized polyester waste 35. The mixture 35 is then sent to a filter 40. The decolorizing reactor unit may also be operated at a temperature ranging from 25° C. to 60° C.

The filter 40 is configured to separate the decolorized polyester waste 50 from the coloured solvents 60. The coloured solvent 60 exiting the filter 40 is then routed to a distillation column 140 to recycle clean organic solvents 160. The distillation column may be performed at a temperature ranging from 40 to 100° C.

The decolorized polyester waste 50 is routed to a depolymerizing reactor unit 90, wherein the reactor is configured to receive a base KOH 80 and a solvent mixture 70 for the depolymerization at a temperature of 20 to 50° C., depending on the type of solvent mixture and polyester used. The volume ratio of the aprotic solvent to the protic solvent is from 1:10 to 100:1. It should be conceived that other first bases, aprotic solvents and protic solvents as disclosed may also be applicable to the system.

The depolymerizing unit 90 discharges an effluent mixture 95 comprising a solid precipitate of mono-salt of dicarboxylic acid and a diol co-product that is miscible with the solvent mixture. The co-product may include ethylene glycol, 1,4-butanediol and mixtures thereof, depending on the type of polyester waste 20 used.

The effluent mixture 95 is then conveyed to a filter 110 to separate the diol and the solvent mixture 120 from the solid monoester salt precipitate, which is transported to the distillation column 140 to recycle the clean solvent mixture 160 and to isolate the co-product diol 240. The precipitated mono-salt of monoester of dicarboxylic acid is retained in the filter 110.

Water 100 is then added to filter 110 to dissolve the mono-salt of monoester of dicarboxylic acid to form an aqueous mono-salt solution 130. It is to be understood that other proper solvent that can dissolve the monoester product may also be applicable in the system. The filter 110 then separates the aqueous mono-salt solution 130 from insoluble polymeric residues. The aqueous mono-salt solution 130 is then conveyed to a filter 150 to remove any remaining solid impurities. The filter 150 contains a microporous membrane wherein a microfiltration process that removes particles higher than 0.08-2 m at a pressure ranging from 7-100 kPa is completed. A clear mono-salt solution 170 is thus obtained.

The mono-salt solution 170 is next transported to an acidifying reactor 200 which is configured to receive a sulfuric acid 190 to adjust pH of the mono-salt solution to 0.1 to 3 to precipitate a solid product comprising the monoester of dicarboxylic acid at room temperature (22° C.). The acidifying reactor 200 discharges a suspension 205 comprising the monoester product 230 and K₂SO₄ solution 220, which is in turn sent to a filter 210 to obtain the final monoester product 230 and an aqueous K₂SO₄ solution 220. The separated K₂SO₄ solution 220 may be stored or recycled for use as a fertilizer. It is to be understood that the salt generated in the acidifying unit depends on the type of base used in the depolymerizing unit 90 and the acid added to the acidifying unit 200.

In an alternative embodiment, a KOH or NaOH solution 180 is optionally added to the reactor 200 for further hydrolysis of the mono-salt to form a dicarboxylic salt. In this case, the sulfuric acid 190 then serves to acidify the dicarboxylic salt to precipitate a solid dicarboxylic acid instead. In this embodiment, the reactor 200 may be operated at substantially the same conditions as described above, e.g., at temperatures from 18° C. to 40° C. It is to be understood that other second bases as disclosed in this invention may be applied to the acidifying reactor.

Each of the reactor units 30/90/200 may respectively comprise more than one reactor e.g., a series of reactors. Each reactor may have a volume up to 100 L and may be equipped with mechanical stirrer means.

Advantageously, the disclosed system enables a large-scale production of monoester of dicarboxylic acid or dicarboxylic acid. More advantageously, the solvents used in the process and the co-products can be recycled across the system disclosed.

### EXAMPLES

### General Considerations

Non-limiting examples of the invention and comparative examples will be further described in greater detail by reference to specific Examples, which should not be construed as in any way limiting the scope of the invention.

The methods of calculating the yield, the selectivity and impurity in the following sections relating to the preparation process are summarized below.

The yield is obtained by measuring the weight of the resulted product relative to the theoretical weight of the product if the reactant is fully converted to the desired product in the reaction.

The major impurity in the reactions is dipotassium salt of terephthalic acid (K2-TPA), which results from the hydrolysis of mono-salt of dicarboxylic acid.

The molar ratio of the mono-salt product: K₂-TPA is calculated by the ratio of the integral area of the Ph-H peaks of mono-salt products, such as K-MMT (7.92 ppm and 8.07 ppm), or K-MET (7.92 ppm and 8.08 ppm), or K-MALT (7.87 ppm and 8.04 ppm), to the peak of the Ph-H of K₂-TPA (7.88 ppm for samples in the preparation K-MMT and K-MET, 7.84 ppm for sample in the preparation of K-MALT) in the ¹H-NMR spectrum of the corresponding product.

Deionized (DI) water was obtained from ELGA Purelab Option Water Purification System with DV25 Reservoir. Sulfuric acid (25 wt. %) was prepared with DI water and concentrated sulfuric acid (95-98 wt. %) which was purchased from Avantor Performance Materials.

### PET Polymer Waste Preparation:

Waste PET water bottles of various brands are used as feedstock, as well as waste PET bottles of soft drinks, coloured PET bottles of body soaps and shampoos. The bottles were cut into scraps with a dimension about 1.5×1.5 cm using a pair of scissors. These scraps were dried in air and used without any cleaning or treatment. In principle, all PET waste bottles, films with or without aluminium coating, PVC coating, or multilayer films containing PET layers (such as PET/adhesive/PE) are applicable as a feedstock in this method.

### Solvents and Chemicals

All the solvents and chemicals were purchased from the sources as provided below, and were used as received in all the examples and comparative examples.
Acetonitrile (ACN): >99.5%, Tokyo Chemical Industry.
Tetrahydrofuran (THF): ≥99.9%, VWR Chemicals.
Ethyl ether anhydrous: ≥99.0%, Tedia High Purity Solvents.
Dichloromethane (DCM): ≥99.5%, Avantor Performance Materials.
Methanol: ≥99.9%, VWR Chemicals.
Ethanol absolute: ≥99.98%, VWR Chemicals.
Allyl alcohol: ≥99%, Sigma-Aldrich.
Dimethyl terephthalate (DMT): ≥99.0%, Tokyo Chemical Industry.
Potassium hydroxide (KOH): ≥85.0%, Merck KGaA.
Sodium hydroxyl (NaOH): ≥99.9%, Merck KGaA.
Deuterium oxide (D₂O, 99.96% D): Cambridge Isotope Laboratories
Chloroform-D (Deuterochloroform, CDCl₃, 99.8% D): Cambridge Isotope Laboratories.
Dimethyl sulfoxide-D₆ (DMSO-D₆, 99.9% D): Cambridge Isotope Laboratories.

### Nuclear Magnetic Resonance (NMR) Measurement:

¹H-NMR (400 MHz) and ¹³C-NMR (100 MHz) spectra were recorded with Bruker (Germany) 400 MHz NMR spectrometer. ¹H-NMR (500 MHz) and ¹³C-NMR (125 MHz) spectra were recorded with Jeol (Japan) 500 MHz NMR spectrometer.

### 1. Conversion of PET to Monoester of Terephthalic Acid

### 1.1. Example 1: Production of MMT from Waste PET Bottles with THF as a Co-Solvent (Methanol: THF=1:1 Volume)

5 g PET scraps (repeating ethylene terephthalate unit 26.02 mmol), 15 mL THF, 15 mL methanol and 1.46 g KOH (26.02 mmol) were added into a 100 mL round bottom flask in order and in air. The resulting mixture was heated to 55° C. by immersing the reactor into a silicon oil bath under stirring. A white slurry was obtained after 1 hour run. The white solid was collected by filtration and washed with 10 mL methanol. A sample was taken, dried in oven at 60° C., and characterized by ¹H-NMR, which indicated that the white solid product is K-MMT. The purity of the crude K-MMT is 99.0%.

For the filtrate, removal of solvents (methanol and THF) from the filtrate with a rotary evaporator at 40° C. generated the crude DMT, which was quickly washed with DI water (5 mL) and methanol (2 mL). The obtained DMT was dried in oven at 60° C. and had a mass of 0.64 g.

The obtained K-MMT was dissolved into 50 mL DI water. Filtration was conducted with a glass filter funnel (G4 sand core) to isolate the unreacted PET (0.09 g), and a clear solution of K-MMT. The aqueous solution of K-MMT was then acidified with H₂SO₄ (25 wt. %) until the pH reaches 2. During the acidification process, MMT was formed immediately and precipitated as white solid, which was isolated by filtration and washed with 40 mL DI water with a glass filter funnel (G4 sand core). The filtered white solid was dried in oven at 60° C., and then characterized with ¹H-NMR and ¹³C-NMR. 3.38 g product was obtained with a yield of 72% (based on a theoretical product of 4.69 g if PET is fully converted to MMT).

¹H-NMR (400 MHz, D₂O, ppm) of K-MMT: 3.96 (s, 3H, —CH₃), 7.92 (d, 2H, ph-H, J_{C-H}=8 Hz), 8.07 (d, 2H, ph-H, J_{C-H}=8 Hz).

¹H-NMR (400 MHz, CDCl₃, ppm) of MMT: 3.96 (s, 3H, —CH₃), 8.13 (d, 2H, ph-H, J_{C-H}=8 Hz), 8.18 (d, 2H, ph-H, J_{C-H}=12 Hz).

¹³C-NMR (100 MHz, DMSO-d₆, ppm) of MMT: 52.44, 129.33, 129.58, 133.15, 134.84, 165.61, 166.55.

The reaction conditions and results are summarized as example 1 in Table 1.

### 1.2. Example 2: Production of MMT from Waste PET Bottles with ACN as a Co-Solvent (Methanol: ACN=1:1 Volume)

The experiment was performed with the same procedure as described in section 1.1, except that 15 mL acetonitrile was used to replace THF. The depolymerization was run for 1 hour affording a white slurry. The purity of K-MMT is 98.5%. MMT was also characterized with ¹H and ¹³C-NMR. 3.51 g MMT was obtained with a yield of 75% (based on a theoretical product of 4.69 g if PET is fully converted to MMT).

The reaction conditions and results are summarized as example 2 in Table 1.

### 1.3. Example 3: Production of MMT from Waste PET Bottles with DCM as Co-Solvent (Methanol: DCM=1:1 Volume)

The experiment was performed with the same procedure as described in section 1.1, except that 15 mL DCM was used to replace THF, and the oil bath was set at 40° C. rather than 55° C. The depolymerization was run for 1 hour affording a white slurry. Purity of K-MMT is 99.3%. MMT was also characterized with ¹H and ¹³C-NMR. 3.09 g MMT was obtained with a yield of 66% (based on a theoretical product of 4.69 g).

The reaction conditions and results are summarized as example 3 in Table 1.

### 1.4. Example 4: Production of Monoethyl Terephthalate (MET) from Waste PET Bottles with DCM as Co-Solvent (Ethanol: DCM=1:1 Volume)

5 g PET scraps (ethylene terephthalate repeating unit is calculated as 26.02 mmol), 15 mL DCM, 15 mL ethanol and 1.46 g KOH (26.02 mmol) were added into a 100 mL round bottom flask in order and in air. The resulting mixture was heated to 40° C. in a silicon oil bath while stirring. The depolymerization was run for 2 hours affording a white slurry. The white solid was collected by filtration. A sample was taken and dried in oven at 60° C. for the characterization by ¹H and ¹³C-NMR, which indicated that the white solid product was K-MET (purity 96.6%).

The obtained K-MET was dissolved into 20 mL DI water. After filtration, 50 mg unreacted PET was isolated. The filtrate is aqueous K-MET solution, which was acidified with H₂SO₄ (25 wt. %) till pH=2. MET was formed immediately and precipitated as a white solid, which was isolated by filtration, washed with 40 mL DI water and dried in oven at 60° C. 3.68 g MET was obtained with a yield of 73% (based on a theoretical product of 5.06 g if PET was fully converted to MET).

¹H-NMR (400 MHz, D₂O, ppm) of K-MET: 1.40 (t, 3H, CH₃, J_{C-H}=8 Hz), 4.42 (q, 2H, CH₂, J_{C-H}=8 Hz), 7.92 (d, 2H, ph-H, J_{C-H}=8 Hz), 8.08 (d, 2H, ph-H, J_{C-H}=8 Hz).

¹³C-NMR (100 MHz, D₂O, ppm) of K-MET: 13.77, 62.80, 129.10, 129.67, 132.14, 141.47, 169.03, 175.11.

The reaction conditions and results are summarized as example 4 in Table 1.

### 1.5. Example 5: Production of MET from Waste PET Bottle with DCM as Co-Solvent (Ethanol: DCM=1:4 Volume)

The experiment was performed with the same procedure as described in section 1.4, except that the volume ratio of ethanol and DCM was altered to 1:4 (6 mL ethanol and 24 mL DCM). The depolymerization was run for 2 hours affording a white slurry. The purity of K-MET product was 96.6%. 3.66 g MET was obtained with a yield of 73% (based on a theoretical product of 5.06 g).

The reaction conditions and results are summarized as example 5 in Table 1.

### 1.6. Example 6: Production of Monoallyl Terephthalate (MALT) from Waste PET Bottles with DCM as Co-Solvent (Allyl Alcohol: DCM=1:2 Volume)

The experiment was performed with the same procedure as described in section 1.4, except that 10 mL allyl alcohol and 20 mL DCM (1:2 volume) were used as the solvent mixture. The depolymerization was run for 4 hours affording a white slurry. The purity of K-MALT was 93.9%. After acidification by H₂SO₄, MALT (3.3 g) was obtained as white powder. The yield was 62% (based on a theoretical product of 5.37 g).

¹H-NMR (500 MHz, D₂O, ppm) of K-MALT: 4.82-4.84 (m, 2H, CH₂), 5.30-5.32 (m, 1H, ═CH₂), 5.40-5.43 (m, 1H, ═CH₂), 6.02-6.10 (m, 1H, ═CH), 7.87 (d, 2H, ph-H, J_{C-H}=8.5 Hz), 8.04 (d, 2H, ph-H, J_{C-H}=8.5 Hz).

¹H-NMR (500 MHz, CDCl₃, ppm) of MALT: 4.83-4.84 (m, 2H, CH₂), 5.28-5.31 (m, 1H, ═CH₂), 5.39-5.43 (m, 1H, ═CH₂), 5.99-6.06 (m, 1H, ═CH), 8.12-8.16 (m, ph-H).

¹³C-NMR (125 MHz, CDCl₃, ppm) of MALT: 66.17, 118.86, 129.81, 130.27, 131.91, 133.07, 134.78, 165.44, 170.27.

The reaction conditions and results are summarized as example 6 in Table 1.

TABLE 1
Production of MMT, MET and MALT from PET waste.^{a}

| | | Co- | Temp | Time^{c} | | Yield |
|---|---|---|---|---|---|---|
| Examples | Alcohol/Volume | solvent/Volume | (° C.)^{b} | (h) | Selectivity ^{d} | (%)^{e} |
| 1 | Methanol/15 mL | THF/15 mL | 55 | 1 | 99.0/1.0 | 72 |
| 2 | Methanol/15 mL | ACN/15 mL | 55 | 1 | 98.5/1.5 | 75 |
| 3 | Methanol/15 mL | DCM/15 mL | 40 | 1 | 99.3/0.7 | 66 |
| 4 | Ethanol/15 mL | DCM/15 mL | 40 | 2 | 96.6/3.4 | 73 |
| 5 | Ethanol/6 mL | DCM/24 mL | 40 | 2 | 96.6/3.4 | 73 |
| 6 | Allyl alcohol/10 mL | DCM/20 mL | 22 | 4 | 93.9/6.1 | 62 |

^{a}Reactions were run in a 100 mL round bottom flask with 5 g PET scraps (repeating unit 26.02 mmol). Equimolar KOH (26.02 mmol) was used.
^{b}Temperature of oil bath.
^{c}The time refers to the depolymerization time to obtain the mono-salt product.
^{d} Calculated with ¹H-NMR.
^{e}Isolated yield of MMT/MET/MALT after acidified with H₂SO₄ (25 wt. %).

It is demonstrated that the solvent mixture can efficiently transform PET into several types of the monoester of terephthalic acid while maintaining high selectivity and yield comparable to the conversion from DMT to monoester.

### 1.7. Examples 7-11: The Effect of Excess KOH in the Production of K-MMT

5 g PET scraps (ethylene terephthalate repeating unit 26.02 mmol), 15 mL DCM, 15 mL methanol and a certain amount of excess KOH were added into a 100 mL round bottom flask in order and in air. The resulting mixture was heated and stirred at a temperature of 40° C. in a silicon oil bath. A white slurry was obtained after a certain time interval as shown in Table 2 below. The white solid was collected by filtration and washed with 10 mL methanol. A sample was taken, dried in oven at 60° C., and characterized with ¹H-NMR, which indicated that the white solid product is K-MMT. The purity of the K-MMT is calculated from ¹H-NMR spectrum.

The crude K-MMT was dissolved into 50 mL DI water. Then a filtration was conducted with a glass filter funnel (G4 sand core) to isolate the unreacted PET and a clear solution of K-MMT. The K-MMT aqueous solution was removed of water with a rotary evaporator at room temperature, affording a white powder product K-MMT, which was dried in oven at 60° C. overnight. The amount of KOH used and results are summarized as examples 7-11 in Table 2.

TABLE 2
Effect of excess KOH at 22° C. and 40°
C. in the production of K-MMT.^{a}

| | KOH:PET | | | | |
|---|---|---|---|---|---|
| | (repeating | | | K-MMT:K₂- | |
| | unit) | | | TPA | Yield |
| | (mole | Temp | Time | (molar | of K-MMT |
| Examples | ratio) | (° C.)^{b} | (h) | ratio) ^{c} | (%)^{d} |
| 7 | 1:1 | 40 | 1 | 99.3/0.7 | 67 |
| 8 | 1:1 | 22 | 1 | 99.0/1.0 | 49 |
| 9 | 1.5:1 | 40 | 1 | 98.3/2.7 | 88 |
| 10 | 1.5:1 | 40 | 0.5 | 98.5/2.5 | 87 |
| 11 | 1.5:1 | 22 | 1 | 98.8/2.2 | 81 |

^{a}Reactions were run in a 100 mL round bottom flask with 5 g PET scraps (repeating unit 26.02 mmol). Methanol (15 mL) and DCM (15 mL) were used.
^{b}Temperature of oil bath.
^{c} Calculated with ¹H-NMR.
^{d}Isolated yield of K-MMT by removing water on a rotary evaporator.

The input of an excess of KOH in the reaction gives rise to the yield of K-MMT with slightly reducing selectivity.

### 2. Conversion of Dimethyl Terephthalate (DMT) to Monomethyl Terephthalate (MMT)

### 2.1. Comparative Example 1: Monohydrolysis of DMT in Pure Methanol

To a 100 mL round bottom flask, 20 mmol DMT, 60 mL methanol, and 20 mmol KOH were added. The mixture was stirred under refluxing condition (65° C.) for 3.5 hours. After the reaction, the flask was cooled down by immersing in tap water for about 3 minutes before filtering the precipitated white slurry with a glass filter funnel (G4 sand core for filtration of fine precipitates with a dimension of 4-7 microns). The obtained white solid was washed with 20 mL dichloromethane (DCM) for 2 times and dried in oven at 60° C. overnight. The white powder product (crude potassium salt of monomethyl terephthalate (K-MMT)) was characterized by ¹H-NMR.

### 2.2. Comparative Example 2: Monohydrolysis of DMT in Pure Methanol

To a 100 mL round bottom flask, 20 mmol DMT, 30 mL methanol, and 20 mmol KOH were added. The mixture was stirred at 40° C. (by heating in water bath) for 1 hour. After the reaction, the flask was cooled by immersing in tap water for about 3 minutes before the precipitated white slurry was filtered. The obtained white solid was washed with 20 mL DCM for 2 times and dried in oven at 60° C. overnight. The white powder product (crude K-MMT) was analyzed with ¹H-NMR.

### 2.3. Examples 12-24: Monohydrolysis of DMT in an Organic Solvent Mixture of DCM and Methanol

To a 100 mL round bottom flask, 20 mmol DMT, 30 mL solvent mixture of DCM and methanol with varying volume ratio, and a desirable amount of KOH were added. The mixture was stirred at 40° C. or 22° C. (room temperature) for 30 minutes or 1 hour. After reaction, the flask was cooled by immersing in tap water for about 3 minutes in case of reaction temperature at 40° C., then the white slurry was filtered, the white solid was washed with 20 mL DCM for two times and dried in oven at 60° C. overnight. The white powder product was analyzed with ¹H-NMR. The volume ratio of DCM to methanol, amount of KOH, reaction time, temperature as well as the yield and impurity analysis are summarized in Table 3 below as examples 12-24.

¹H-NMR (400 MHz, D₂O, ppm) of K-MMT: 3.96 (s, 3H, —CH₃), 7.92 (d, 2H, ph-H, J_{C-H}=8 Hz), 8.07 (d, 2H, ph-H, J_{C-H}=8 Hz).

TABLE 3
Conversion of DMT to MMT

| | DCM:Methanol | DMT/KOH | | | | K-MMT/ | Impurity at |
|---|---|---|---|---|---|---|---|
| | (volume | Molar | Temp | Time | Yield | K₂-TPA | 7.88 ppm |
| Examples | ratio) | ratio | (° C.) | (h)^{a} | (%)^{b} | (molar ratio)^{c} | (in ¹H-NMR) |
| 12 | 1:1 | 1:1 | 40 | 1 | 75 | 99.6/0.4 | Weak |
| 13 | 2:1 | 1:1 | 40 | 1 | 80 | 99.8/0.2 | Weak |
| 14 | 4:1 | 1:1 | 40 | 1 | 82 | 99.8/0.2 | Minor |
| 15 | 6:1 | 1:1 | 40 | 1 | 84 | 99.9/0.1 | Minor |
| 16 | 8:1 | 1:1 | 40 | 1 | 84 | 99.9/0.1 | Minor |
| 17 | 10:1 | 1:1 | 40 | 1 | 84 | 99.9/0.1 | None |
| 18 | 10:1 | 1:1 | 40 | 0.5 | 84 | 99.9/0.1 | None |
| 19 | 14:1 | 1:1 | 40 | 1 | 84 | 99.6/0.3 | None |
| 20 | 20:1 | 1:1 | 40 | 1 | 83 | 97.9/2.1 | None |
| 21 | 30:1 | 1:1 | 40 | 1 | 80 | 93.7/6.3 | None |
| 22 | 60:1 | 1:1 | 40 | 1 | 71 | 81.0/19.0 | None |
| 23 | 10:1 | 1:1 | 22 | 1 | 84 | 99.9/0.1 | None |
| 24 | 10:1 | 1:1.2 | 22 | 1 | 92 | 99.4/0.6 | Minor |
| Comparative | N.A.^{d} | N.A. | reflux | 3.5 | 67 | 98.1/1.9 | Strong |
| Example 1 | | | | | | | |
| Comparative | N.A.^{e} | N.A. | 40 | 1 | 28 | 98.2/1.8 | Strong |
| Example 2 | | | | | | | |

Note:
20 mmol DMT was used in all the examples 12-24 and comparative examples 1&2. For experiments 12-24, 30 mL solvent mixture was used.
^{a}The time refers to the reaction time of monohydrolysis to obtain the K-MMT.
^{b}Yields are isolated white powder K-MMT.
^{c}The ratio of K-MMT to K₂-TPA were calculated from ¹H-NMR.
^{d}Use 60 mL methanol as solvent.
^{e}Use 30 mL methanol as solvent.

As shown in Table 3, the monohydrolysis reaction of DMT is slow with unsatisfactory selectivity in pure methanol (Comparative examples). Even when running at refluxing temperature for 3.5 hours, the isolated yield was 67% with a K-MMT/K₂-TPA molar ratio of 98.1:1.9 (Comparative Example 1). Comparative Example 2, which was performed at lower temperature (40° C.), gave a comparable K-MMT/K₂-TPA molar ratio of 98.2:1.8. Therefore, the selectivity of the monohydrolysis reaction undertaken in pure methanol is not affected by temperature, but the isolated yield decreased from 67% to 28% due to the shortened reaction time from 3.5 hours to 1 hour and the lower temperature.

To our surprise, when replacing half of methanol with DCM in Comparative Example 2, it is found that, at 40° C., the yield significantly increased from 28% to 75%, the molar ratio of K-MMT/K₂-TPA increased to 99.6:0.4 from 98.2:1.8, and the impurity present at 8.12 ppm of ¹H-NMR spectrum became much weaker (Example 12).

More methanol was replaced with DCM for the process to obtain K-MMT, while the total volume of the organic solvent mixture remains 30 mL. The results with DCM/methanol volume ratio from 2:1 to 60:1 was shown in Table 3 (Examples 13-22).

Compared to Example 12, when DCM/methanol volume ratio was set at 2:1 (Example 13) or 4:1 (Example 14), the K-MMT/K₂-TPA molar ratio increased to 99.8:0.2. When DCM/methanol volume ratio was set at 6:1, 8:1 or 10:1 (examples 15-17), the K-MMT/K₂-TPA molar ratio increased to 99.9:0.1 and the yield also increased to 84%. Especially, the reaction using DCM/methanol volume ratio of 10:1 (Examples 17 and 18) showed extremely high K-MMT/K₂-TPA molar ratio (99.9:0.1) and yield of 84% even within 30 min reaction time. When further increasing the volume ratio of DCM/methanol to 14:1 (example 19), the K-MMT/K₂-TPA molar ratio slightly decreased to 99.4:0.6. At very high-volume ratio of DCM/methanol (Examples 20-22: 20:1, 30:1, 60:1), the K-MMT/K₂-TPA molar ratio decreased to 97.9%, 93.7% and 81.0%, respectively. The yield also dropped to 83%, 80% and 71%, respectively. Meanwhile, the impurity at 8.12 ppm became weaker with the increasing volume ratio of DCM/methanol and it even disappeared when DCM/methanol volume ratio is above 10:1.

For Examples 12-18, especially examples 14-18, it was observed that DMT was quickly dissolved in the organic solvent mixture upon stirring compared to using pure methanol as solvent. This may account for the high reaction rate. It was also observed during the experiments that with the increasing of DCM/methanol volume ratios, the dissolution of KOH became slower, leading to a high DMT/KOH molar ratio in the reaction mixture during the hydrolysis. This may be one of the reasons for the high selectivity when DCM/methanol volume ratio is ranging from 1:1 to 14:1. However, at very high DCM/methanol volume ratios, e.g., above 20:1, the solubility of KOH in the solvent mixture became very limited, which may account for the slower reaction rate, lower selectivity and yield.

Surprisingly, if the reaction time was shortened from 1 hour to 0.5 hour as demonstrated by Example 18 with DCM/methanol volume ratio at 10:1, it exhibits the same selectivity and yield as Example 17. When the reaction temperature was adjusted to room temperature, i.e. 22° C. (Example 23), a high selectivity at 99.9% and yield at 84% are retained. Further, when the volume ratio of DMT/KOH decreased to 1:1.2 by using excess KOH, and the reaction was also performed at 22° C. (Example 24), the yield increased to 92% with a slight decrease in selectivity to 99.4%.

### 2.4. Examples 25-27: Monohydrolysis of DMT in an Organic Solvent Mixture of Tetrahydrofuran (THF)/Acetonitrile (ACN)/Diethyl Ether and Methanol

The reaction was performed with the same method as described in 2.3 in which the DCM was replaced by THF, or ACN, or diethyl ether. The reaction temperature, volume of the solvents, temperature and yield of examples 25-27 are summarized in Table 4.

TABLE 4
Results of monohydrolysis of DMT using THE, ACN and diethyl ether as co-solvent.

| | | | Solvent | | | K-MMT/ | Impurity at |
|---|---|---|---|---|---|---|---|
| | | Solvent/methanol | mixture | Temp | Yield | K₂-TPA | 7.88 ppm |
| Examples | Solvent | (volume ratio) | (mL) | (° C.) | (%)^{a} | (molar ratio)^{b} | (in ¹H-NMR) |
| 25 | THF | 10:1 | 40 | 40 | 85 | 99.9/0.1 | Minor |
| 26 | ACN | 10:1 | 60 | 40 | 84 | 99.9/0.1 | Minor |
| 27 | Ether | 10:1 | 60 | 30 | 83 | 99.9/0.1 | Minor |

Note:
20 mmol DMT and equivalent KOH were used in examples 25-27.
^{a}Yields are isolated white powder K-MMT.
^{b}The molar ratio of K-MMT to K₂-TPA was calculated from ¹H-NMR.

The results show that reactions using the three co-solvents in the solvent mixture enable a high K-MMT/K₂-TPA molar ratio (99.9:0.1) with a minor peak of impurity.

### 3. Example 28: Acidification of K-MMT with H₂SO₄ (25 wt. %)

To a 500 mL round bottom flask equipped with a mechanical stirrer, 5 g K-MMT and 60 mL water were added. A clear solution was formed after stirring at room temperature. Then H₂SO₄ (25 wt. %) was added dropwise while stirring until pH of the solution reaches 1˜2. The white slurry was filtered and washed with water (40 mL). The product was dried in oven at 60° C. overnight. 4.04 g MMT was obtained with a yield 98%. The product was analyzed with ¹H and ¹³C-NMR.

¹H-NMR (400 MHz, CDCl₃, ppm) of MMT: 3.96 (s, 3H, —CH₃), 8.13 (d, 2H, ph-H, J_{C-H}=8 Hz), 8.18 (d, 2H, ph-H, J_{C-H}=12 Hz).

¹³C-NMR (100 MHz, DMSO-d₆, ppm) of MMT: 52.44, 129.33, 129.58, 133.15, 134.84, 165.61, 166.55.

### 4. Example 29: Conversion of K-MMT to TPA

To a 100 mL round bottom flask, K-MMT (2.84 g, 13.01 mmol), KOH (0.875 g, 15.61 mmol) (or NaOH, 0.624 g, 15.61 mmol) and deionized water (40 mL) were added and stirred to form a solution. A terephthalic salt (K₂-TPA) was obtained by hydrolysing K-MMT in the presence of KOH or NaOH. After stirring for 10 min at room temperature, the solution was acidified by H₂SO₄ (25 wt. %) till pH reaches 2. The as formed white powder product TPA was collected by filtration, washed with 10 mL DI water for two time (2×10 mL), dried in oven at 60° C. 2.10 g TPA was obtained with a yield 97%.

### 5. Example 30: Scale-Up Production of MMT from Waste PET Bottles with DCM as Co-Solvent (Methanol: DCM=1:1 Volume)

To a 5-liter DURAN 3-neck reactor equipped with an overhead mechanical stirrer, PET scraps (150.15 g, 781.34 mmol), KOH (43.84 g, 781.34 mmol), DCM (450 mL) and methanol (450 mL) were added. The mixture was heated in a water bath (40° C.) under vigorous stirring. After 1 hour reaction, the mixture was transformed to a white slurry. The crude K-MMT was isolated by filtration, washed with 200 mL methanol for two times (2×200 mL), filtered again to give a product shaped like a white wet cake. A sample of the product was taken to be dried at 60° C. for ¹H-NMR analysis, which showed that the purity of K-MMT was 98.5%.

For the clear filtrate, removal of the solvent (DCM and methanol) with a rotary evaporator at 40° C. gave a pale, yellow solid, which was washed with 40 mL DI water, filtered and quickly washed with methanol (10 mL). The obtained white DMT was dried at 60° C., which was then characterized with ¹H-NMR. The weight of DMT is 12.6 g.

The obtained white cake (crude K-MMT) was dissolved into 2.0 L DI water. After filtration, unreacted PET (7.0 g) was isolated. The clear K-MMT solution was acidified with 25 wt. % H₂SO₄ till pH of the solution reached 2, resulting in the immediate formation of MMT as a white slurry. The product MMT was isolated by filtration, washed with DI water (400 mL), dried in oven at 60° C. 106.58 g MMT was obtained with a yield of 76% (based on a theoretical product of 140.77 g if PET wastes were fully converted to MMT).

### 6. Example 31: Scale-Up Production of the Monohydrolysis of DMT with Solvent Mixture DCM/Methanol (10:1)

To a 5-Liter DURAN 3-neck reactor equipped with an overhead mechanical stirrer, DMT (233.03 g, 1.2 mol), KOH (67.33 g, 1.2 mol), DCM (1.64 L) and methanol (164 mL) were added. The mixture was vigorously stirred at 22° C. for 1 hour. The resulted white slurry was then filtered to afford a white cake which was washed with DCM (400 mL). The obtained white powder product was dried at 60° C. 224.5 g white powders were obtained with a yield of 86%. The ¹H-NMR showed that the purity of K-MMT was 99.9%.

### INDUSTRIAL APPLICABILITY

The disclosed process may be used for depolymerizing a dicarboxylate polyester into a mono-salt of the dicarboxylic acid. The disclosed process may also be used for the production of a monoester of dicarboxylic acid or dicarboxylic acid. Both products are valuable materials for application in a variety of industries such as pharmaceutical industry and polymer industry. In particular, the process disclosed may be used for the conversion of diester of dicarboxylic acid, regardless of the source of said diester, e.g., terephthalic polymer wastes, product from esterification of terephthalic acid, etc. Furthermore, the process disclosed may be used for upcycling of polyester wastes from consumer products such as water bottles, shampoo bottles, textiles etc. The reaction condition at ambient environment and short reaction time allows a scalable production of said monoester.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A process of depolymerizing a polyester of a dicarboxylic acid, the process comprising:
depolymerizing the polyester in the presence of a first base and a solvent mixture to yield a product comprising a mono-salt of a monoester of said dicarboxylic acid;
wherein said solvent mixture comprises at least one aprotic solvent and at least one protic solvent; and
wherein the aprotic solvent and protic solvent are provided in a volume ratio of from 1:10 to 100:1.

2. The process of claim 1, wherein the polyester is selected from the group consisting of polyethylene terephthalate (PET), polybutylene terephthalate (PBT), polytrimethylene terephthalate (PTT), polyethylene naphthalate (PEN), block copolymer of forgoing polyesters, substituted foregoing polyesters, branched forgoing polyesters and mixtures thereof.

3. The process of claim 1, wherein the aprotic and protic solvents are provided in a volume ratio of 1:1 to 15:1.

4. The process of claim 1, wherein the aprotic solvent comprises at least one polar aprotic solvent selected from the group consisting of acetonitrile, tetrahydrofuran, acetone, dimethyl formamide, and dimethyl sulfoxide; and/or at least one non-polar aprotic solvent selected from the group consisting of toluene, dichloromethane, chlorobenzene, xylene, diethyl ether, and combinations thereof;
and/or the protic solvent is selected from the group consisting of methanol, ethanol, 2-fluoroethanol, 2-chloroethanol, 2,2,2-trichloroethanol, n-propanol, isopropanol, n-butanol, tert-butanol, allyl alcohol, propargyl alcohol, 2-aminoethanol, 2-dimethylaminoethanol, ethylene glycol, propylene glycol, 1,4-butanediol, and mixtures thereof,
optionally wherein the aprotic solvent is selected from the group consisting of tetrahydrofuran, acetonitrile, dichloromethane, diethyl ether, and mixtures thereof,
and optionally wherein the protic solvent is selected from the group consisting of methanol, ethanol, allyl alcohol and mixtures thereof.

5-7. (canceled)

8. The process of claim 1, wherein the mole ratio of the first base to a repeating monomer unit of the polyester is from 0.5 to 2.

9. The process of claim 1, wherein the mole ratio of the first base to a repeating monomer unit of the polyester is from 1 to 1.5.

10. The process of claim 1, wherein the first base comprises at least one inorganic base, at least one organic base, or a mixture thereof.

11. The process of claim 10, wherein the inorganic base is selected from the group consisting of alkali metal hydroxide, alkaline earth metal hydroxide, ammonium hydroxide, and mixtures thereof;
and/or the organic base is selected from the group consisting of metal salt of methoxide, metal salt of ethoxide, metal salt of n-propoxide, metal salt of iso-propoxide, metal salt of n-butoxide, metal salt of tert-butoxide, and mixtures thereof,
optionally wherein the inorganic base is selected from the group consisting of potassium hydroxide, sodium hydroxide, and mixtures thereof,
optionally wherein the metal in the organic base is selected from alkali metal or alkaline earth metal,
and optionally wherein the organic base is selected from the group consisting of potassium methoxide, potassium ethoxide, potassium n-propoxide, potassium iso-propoxide, potassium n-butoxide, potassium tert-butoxide, sodium methoxide, sodium ethoxide, sodium n-propoxide, sodium iso-propoxide, sodium n-butoxide, sodium tert-butoxide, and mixtures thereof.

12.-15. (canceled)

16. The process of claim 1, wherein said depolymerizing is performed at a temperature ranging from 10° C. to 90° C.;
and/or said depolymerizing is undertaken for a time period from 10 minutes to 10 hours.

17. The process of claim 1, wherein said depolymerizing is performed at a temperature ranging from 22° C. to 55° C.

18. The process of claim 1, wherein said depolymerizing is undertaken for a time period from 30 minutes to 5 hours.

19. The process of claim 1, further comprising separating the mono-salt product as solid precipitates.

20. The process of claim 19, further comprising reacting the mono-salt product with an acid solution to produce a monoester of said dicarboxylic acid, wherein the acid solution is selected from the group consisting of hydrochloric acid (HCl), sulfuric acid (H₂SO₄), phosphoric acid (H₃PO₄), acetic acid, formic acid, citric acid and mixtures thereof.

21. The process of claim 20, wherein the mono-salt product is acidified until a pH value of from 0.1 to 3; and/or wherein said reacting the mono-salt product with an acid solution, is performed at a temperature ranging from 18° C. to 40° C.

22. (canceled)

23. The process of claim 19, further comprising a step of reacting the mono-salt product with a second base to produce a dicarboxylic salt,
optionally wherein the second base is an inorganic base selected from the group consisting of alkali metal hydroxide, alkaline earth metal hydroxide, ammonium hydroxide, and mixtures thereof,
and optionally wherein the second base is an inorganic base selected from potassium hydroxide, sodium hydroxide, and mixtures thereof.

24.-25. (canceled)

26. The process of claim 23, further comprising reacting the said dicarboxylic salt with an acid solution to produce a dicarboxylic acid, wherein the acid solution is selected from the group consisting of hydrochloric acid (HCl), sulfuric acid (H₂SO₄), phosphoric acid (H₃PO₄), acetic acid, formic acid, citric acid and mixtures thereof.

27. A process for preparing a mono-salt of monoester of terephthalic acid, the process comprising:
hydrolyzing a diester of terephthalic acid in the presence of a first base and a solvent mixture to yield the mono-salt of the monoester of terephthalic acid;
wherein said solvent mixture comprises at least one aprotic solvent and at least one protic solvent; and
wherein the aprotic solvent and protic solvent are provided in a volume ratio of from 1:10 to 100:1.

28. The process of claim 27, wherein the first base is provided to the diester of terephthalic acid in a mole ratio of from 0.5 to 2.5.

29. The process of claim 28, wherein the mole ratio of the first base to the diester of terephthalic acid is from 1 to 1.5.

30. The process of claim 27, wherein the diester is provided at a concentration from 0.1 M to 2 M or preferably 0.1 to 0.7 M.
